(19) United States
(12) Patent Application Publication
(10) Pub. No.: US 2005/0245999 A1
(43) Pub. Date: Nov. 3, 2005

(54) Tanning bed support structure and a tanning bed comprising the same

(76) Inventor: Charles M. Dixon, Beaumont, TX (US)

Correspondence Address: KRAMER & AMADO, P. C.;Suite 240, 1725 Duke Street, Alexandria, VA, 22314, US

(21) Appl. No.: 10/834,871

(22) Filed: Apr. 30, 2004

(51) Int. Cl. A61N 5/06

(52) U.S. Cl. 607/91

(57) ABSTRACT

A framework for supporting a rotatably mounted tanning bed, comprising a support frame; an axle rotatably mounted to said support frame, said axle being adapted to non-rotatably support a tanning bed; and a motor adapted to reversibly rotate the axle from a first position to a second position, wherein said tanning bed is in a horizontal position when the axle is in said first position, and said tanning bed is in a vertical position when the axle is in said second position; and a tanning machine comprising a tanning bed mounted on said framework.

### BACKGROUND OF THE INVENTION

1. Field of the Invention

This invention relates generally to a tanning apparatus, and more particularly to a portable tanning apparatus capable of tanning the entire body of a person. More particularly, the invention relates to a portable apparatus for allowing a person to tan while in either a reclining position or a standing position.

2. Description of Related Art

In our culture, suntanned skin is considered highly desirable and, as a result, each year people willingly expose themselves to the sun for extended periods to obtain a golden or bronze suntan. However, there are certain risks associated with this, as the sun emits a wide range of ultraviolet frequencies, many of which are deleterious to human health. Ultraviolet light having a wavelength of 315 to 380 nanometers (UVA light) is most beneficial for tanning, and is comparatively safe. Other types of ultraviolet light such as light having a wavelength between 280 and 315 nanometers (UVB) or light having a wavelength between 180 and 200 nanometers (UVC) can cause severe sunburn. Of even greater concern is the fact that sunburns during the tanning process greatly increases the risk of skin cancers of various types.

One way of obtaining a tan with reduced risk is through the use of an artificial tanning device. State of the art tanning lamps that provide low levels of UVB radiation have been developed, which are considerably safer than direct sun exposure under uncontrolled conditions. These lamps, in addition to providing a very small amount of the dangerous UVB radiation, give off less heat than conventional lamps. They are shaped like a conventional fluorescent bulb and may be used to expose a person's whole body to UV light. In order to take advantage of the new UVA lamps, tanning devices that are of a sufficient length to provide even radiation to the entire body of a user have been designed.

Two types of tanning devices are in common use, each designed to accommodate the preferences of a particular group of tanning customer. On the one hand, some people prefer to tan in a prone position. The devices typically feature a couch or bed, and a canopy containing UV lights above the couch or bed. The couch or bed is frequently transparent or translucent, so as to allow a second array of UV lights to be positioned below. The couch or bed is frequently contoured to maximize comfort of a customer lying on his back by, for example, elevating the head or knees. This allows the customer to relax during his tanning session. Other customers prefer to tan in a standing position, between at least two vertical arrays of UV lamps. This system allows the person tanning to turn during his tanning session so as to fully expose all sides of his body to the UV light, achieving a more thorough tan.

This leads to a problem for the person running the tanning facility. Since some customers prefer to tan standing up, while others prefer to tan lying down, he is required to provide two types on tanning systems, one which allows a client to lie prone while tanning, and one which allows the client to stand erect while tanning. Accordingly, he is required to spend money on two sets of equipment. Further, he has to pay rent on increased space for storage and maintenance of the two sets of equipment. It would be a great advantage to the proprietor of a tanning establishment if one set of equipment was able to allow his customers to tan in multiple positions, including standing, lying down, or something in between. It would also be advantageous to the proprietor of a tanning establishment and to his customers to allow direct customer control of tanning positions.

Additionally, tanning beds wear out over time and must be replaced. Also, upgrades to tanning beds are commonly made, necessitating tanning bed replacement if the proprietor of a tanning salon wishes to stay competitive. Frequently, the framework supporting the tanning bed remains in excellent condition. Current tanning beds, however, require that the entire bed be replaced. It would be a substantial savings to the proprietor of a tanning salon if the tanning bed could be conveniently removed from this framework and replaced without requiring replacement of the framework itself.

The invention disclosed herein answers the long-felt needs of these tanning salon proprietors and their customers. It provides a tanning bed that may be conveniently rotated from a vertical position to a horizontal position, allowing the proprietor to give his customers the option of tanning in multiple positions with a single set of equipment. Further, it provides a tanning bed mounted on a framework, where the bed may be removed from the framework, greatly easing bed replacement and transportation.

The foregoing objects and advantages of the invention are illustrative of those that can be achieved by the present invention and are not intended to be exhaustive or limiting of the possible advantages that can be realized. Thus, these and other objects and advantages of the invention will be apparent from the description herein or can be learned from practicing the invention, both as embodied herein, or as modified in view of any variation which may be apparent to those skilled in the art. Accordingly, the present invention resides in the novel methods, arrangements, combinations and improvements herein shown and described.

### SUMMARY OF THE INVENTION

In light of the present need of tanning salon proprietors for equipment that allows them to serve both customers who prefer to tan in a standing position and customers who prefer to tan while in a prone position, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

The current invention is a rotatably mounted tanning bed system. The apparatus includes a support frame and an axle rotatably mounted to the support frame. The system further includes a motor and a system of gears. The motor rotates a first gear, and rotation of the first gear directly or indirectly rotates a main gear mounted on the axle. A tanning bed is mounted to the axle so that rotation of the axle causes the tanning bed and the axle to move as a unit.

The tanning bed mounted to the axle comprises a bed adapted to support the weight of an occupant and a canopy facing the bed so that the occupant may position himself between said canopy and said bed. A set of UV lamps is mounted in the canopy, in a position that allows them to irradiate the occupant of the tanning bed. Preferably, a first set of UV lamps is mounted in the canopy, and a second set of UV lamps is mounted beneath the occupant's bed, which is then made out of a material that is transparent or translucent to UV light. The occupant may then be irradiated from multiple directions in a single tanning session.

Preferably, the axle rotates reversibly from a first position to a second position, where the tanning bed is in a horizontal position when the axle is in said first position, and the tanning bed is in a vertical position when the axle is in said second position. This allows customers to tan in a standing position when the tanning bed is in its vertical position, or to tan in a prone position when the tanning bed is in its horizontal position. The motorized rotation of the axle allows the tanning salon proprietor to quickly change the position of the tanning bed depending on his customer's expressed preference. If desired, the tanning bed may also be rotated manually.

In another embodiment, the tanning bed is removably mounted to the axle. This allows the apparatus to be quickly and easily disassembled for transportation, or to replace a worn out or obsolete tanning bed with a new tanning bed while retaining the support framework.

### BRIEF DESCRIPTION OF THE DRAWINGS

In order to better understand the present invention, reference is made to the accompanying drawings, wherein:

FIGS. 1a and 1b show two views of a preferred embodiment of a support framework for a rotatable tanning bed. FIG. 1a shows a view of the support structure holding up the axle upon which the tanning bed rotates. FIG. 1b shows a view of the support structure, together with the drive mechanism for rotating the axle.

FIGS. 2 and 3 show alternative embodiments of the drive mechanism for rotating the axle.

FIGS. 4a and 4b show two views of a tanning bed adapted to be removably mounted to the support structure of FIG. 1a.

FIGS. 5a, 5b and 5c show methods of mounting the tanning bed of FIG. 4a to the axle of the support frame of FIG. 1a so that the axle and the tanning bed rotate as a single unit.

### DETAILED DESCRIPTION OF THE PREFERRED EMBODIMENTS OF THE INVENTION

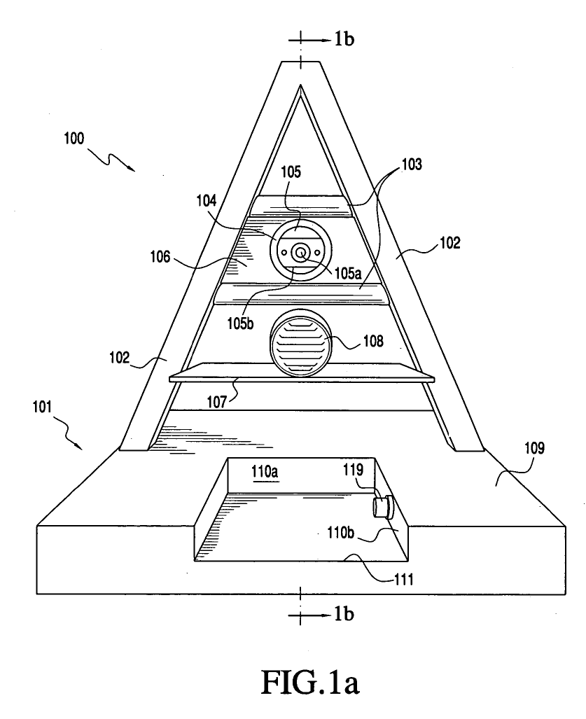

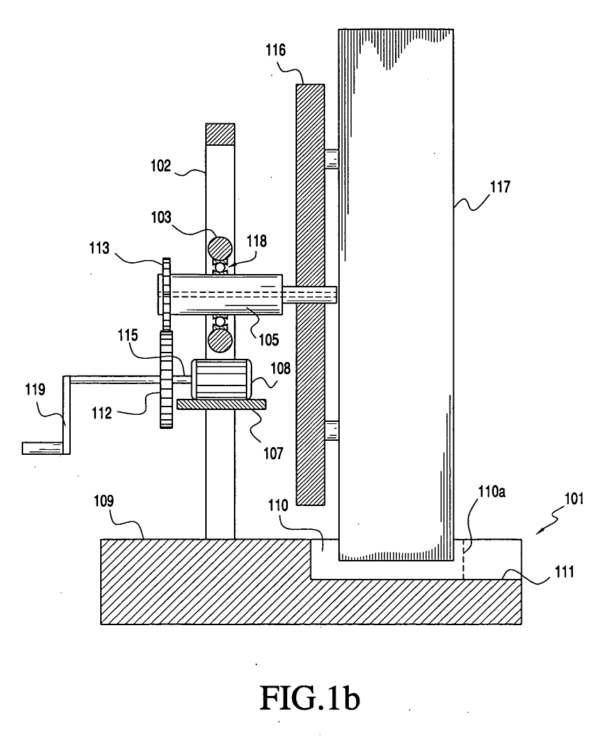

Referring now to the drawings, in which like numerals refer to like components or steps, there are disclosed broad aspects of the preferred embodiments of the present invention. FIGS. 1a and 1b show a framework 100 for supporting a tanning bed, mounted on a base 101. The base 101 includes two slanted support members 102, preferably made from a metal tube or rod. Support members 102 may be made from iron, steel, aluminum, or titanium. Alternatively, members 102 may be made from a high-strength composite polymeric material, such as a fiber-reinforced plastic. For reasons of both weight and cost, however, the preferred material for members 102 is a tubular steel material. Two crossbeams 103 run between members 102, and provide reinforcement for the members 102. An axle-supporting tubular element 104 is mounted between crossbeams 103, and a rotatable axle 105 passes longitudinally through element 104. There is a longitudinal bore 105a through axle 105. The axle is preferably cylindrical as it passes through element 104. After the axle leaves element 104, however, at least one surface 105b of the axle may be flattened, if desired. A flat metal or plastic plate 106 may be positioned between crossbeams 103, with tubular element 104 being fixed to plate 106 as well as to crossbeams 103. This plate 106 provides additional reinforcement to the element 104. A shelf 107 is mounted between supports 102, and a motor 108 is positioned on shelf 107.

Base 101 includes a flat support member 109 to which slanted support members 102 are mounted. A trench or groove 110 is positioned alongside support members 102. When a tanning bed support 116 is mounted onto axle 105, rotation of the axle 105 causes a tanning bed 117 attached to support 116 to rotate from a first position in which the tanning bed is supported above and parallel to support member 109 to a second position in which the tanning bed is oriented vertically (See FIG. 1b). In this second position, trench 110 provides additional clearance for a foot end of the tanning bed, which is rotated into the trench. A portion 110a of the side of trench 110 is cut away, providing a step 111, which allows a person to step into trench 110 when the tanning bed is in its vertical position. The trench additionally has a back wall 110b, which serves to prevent tanning bed 117 from rotating past the vertical position. A pneumatic damper positioned against this back wall 110b serves to reduce the shock of impact between tanning bed 117 and wall 1110b. If desired, rotation of the tanning bed may be stopped at one or more positions intermediate between the first and second positions. These intermediate positions may be preprogrammed into a computer controlling rotation of the axle by the motor. Alternatively, rotation of the axle by the motor may be under direct control of either the tanning salon proprietor or his customer, and rotation of the tanning bed may be stopped at the first position, the second position, or any position therebetween.

FIG. 1b shows a detailed view of one embodiment of a mechanism for rotation axle 105. Motor 108 rotates a shaft 115 on which a spur gear 112 is mounted. Spur gear 112 in turn rotates a second spur gear 113. Gear 113 is rigidly mounted to axle 105, so that axle 105 serves as the gear shaft of gear 113. Thus, rotation of gear 113 rotates both axle 105 and the tanning bed 117 attached thereto. Motor 108 may be used to rotate axle 105 in either a forward or a reverse direction.

FIG. 1b also shows a method of mounting the axle between crossbeams 103 using a radial ball bearing 118. Bearing 118 includes an outer ring fixed to crossbeams 103, an inner ring fixed to axle 105 and a plurality of balls therebetween. The balls are retained between the inner and outer rings by a cage mechanism. The outer ring of the bearing serves as axle-supporting tubular element 104. Bearing 118 thus allows axle 105 to rotate relative to framework 100 with minimal friction. Alternatively, if use of a bearing is not desired, a low friction material may be positioned between axle-supporting tubular element 104, shown in FIG. 1a, and axle 105. This may be done by coating at the inner surface of tubular element 104 with a low-friction fluoropolymer, such as Teflon®, to reduce friction. Alternatively, the outer surface of the portion of axle 105 which passes through element 104 may be coated with Teflon®. More preferably, both the inner surface of tubular element 104 and the outer surface of axle 105 may be coated with Teflon®. Alternatively, tubular element 104 and axle 105 may be left uncoated, and a tubular sleeve of Teflon®, or a Teflon®-coated material (not shown in the figures), may be positioned between 104 and 105.

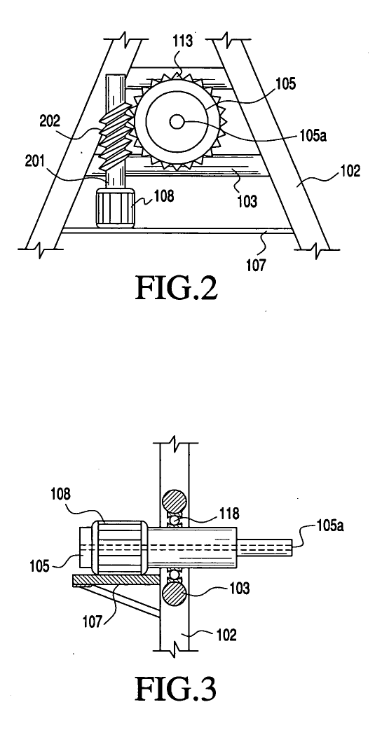

FIG. 2 shows an alternative embodiment of the drive mechanism. The motor 108 disposed on shelf 107 drives a gear shaft 201 that is directed perpendicularly to axle 105. Gear shaft 105 carries a worm gear 202 having a helical thread that meshes with spur gear 113, allowing rotation of the worm gear to drive axle 103. Use of a worm gear 202 in this invention has a significant advantage over use of a spur gear 112. Worm gear meshes are irreversible; when a worm gear is turned, the meshing spur gear will turn, but turning the spur gear will not turn the worm gear. The resulting mesh is “self locking.” This reduces the chance that gear slippage will cause the tanning bed to rotate while it is occupied by a customer. On the other hand, the worm gear operates with low efficiency, since there is comparatively high friction between the worm gear and gear 113. FIG. 3 shows a second alternative embodiment of the drive mechanism. The motor 108 disposed on shelf 107 drives axle 105 directly, without requiring intervening gears.

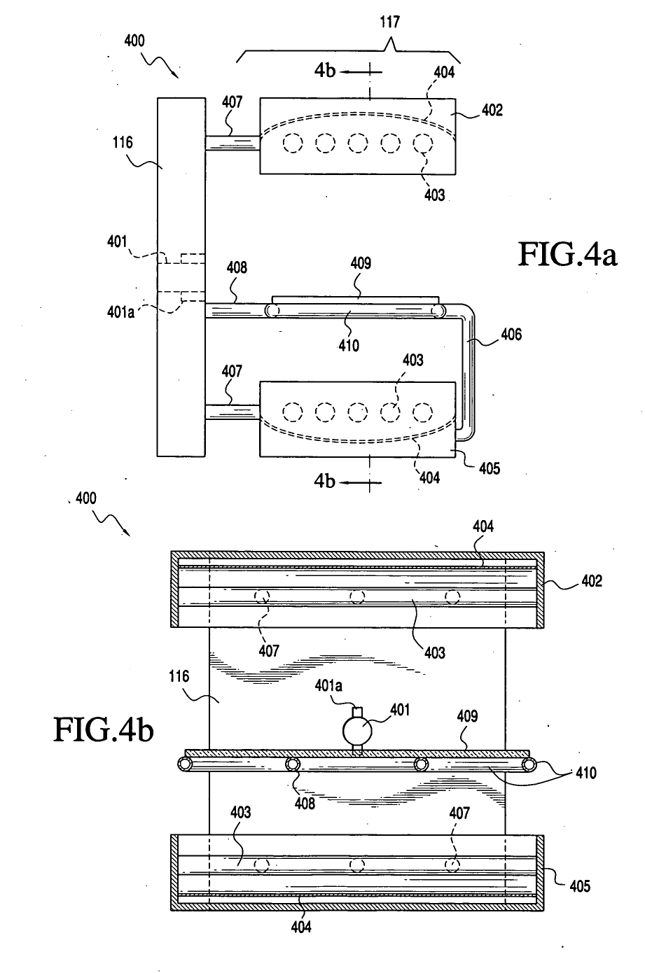

FIGS. 4a and 4b show two views of a tanning bed assembly 400 to be used with the framework of FIG. 1a. The tanning bed assembly 400 includes a tanning bed support 116 and a tanning bed 117 attached to support 116, as shown in FIG. 1b. Support 116 is a rigid element made of metal or a composite material, such as fiber-reinforced plastic. A bore 401 adapted to receive axle 105 runs through support 116, and two radial notches 401a extend from the bore. If desired, the notches 401a may be replaced by threaded holes adapted to receive a metal bolt. The tanning bed 117 comprises a WV-transparent or translucent bed 409 positioned between an upper canopy 402 and a lower base unit 405. Canopy unit 402 and base unit 405 are each designed to direct WV light toward a tanning bed occupant positioned between bed 409 and canopy unit 402. Canopy unit 402 and base unit 405 each include a plurality of WV lamps 403, and a reflector 404, which directs light from the WV lamps 403 toward bed 409. Canopy unit 402 and base unit 405 are each mounted to support 116 by a series of support struts 407. Another series of support struts 408 is used to mount bed 409 to support 116. Bed 409 is supported by an oblong or rectangular bed support 410 made of tubular metal or fiber-reinforced resin elements 410. These elements 410 are then mounted to struts 408. Another series of struts 406 connects bed support 410 to the lower base unit 405. This helps give additional rigidity to the bed. If desired, the assembly may include two banks of WV lights 403 in the upper canopy unit 402, each with its associated reflector 404, where the two banks of lights are arranged at an angle of between 60 and 120 degrees to each other. This allows the tanning bed occupant to achieve a more even tan by exposing himself to light from three directions at once.

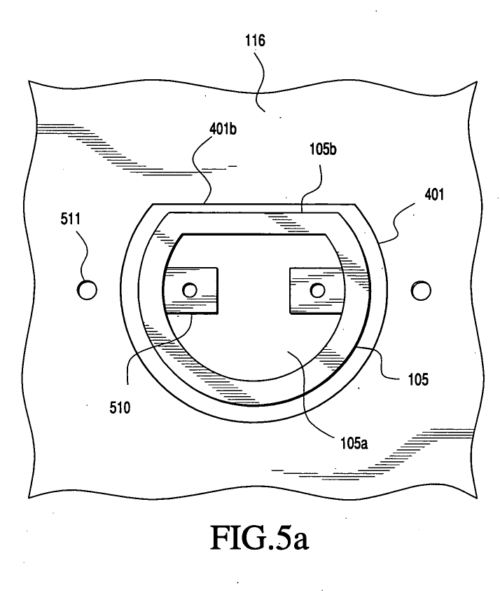

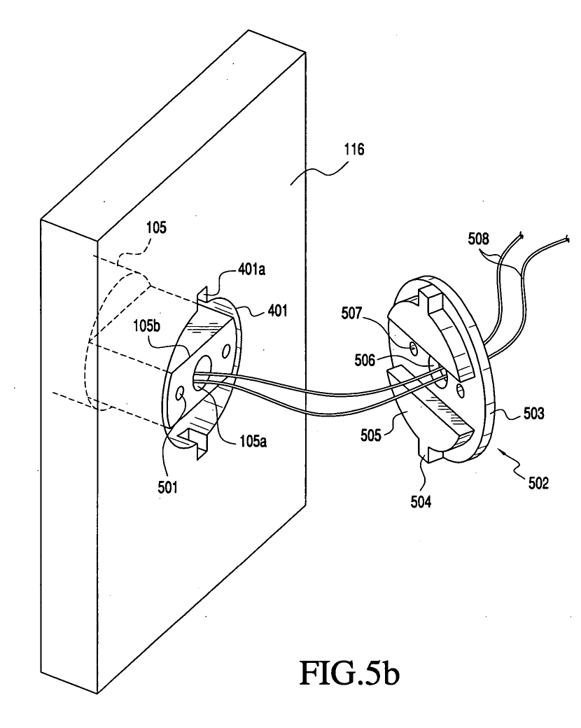

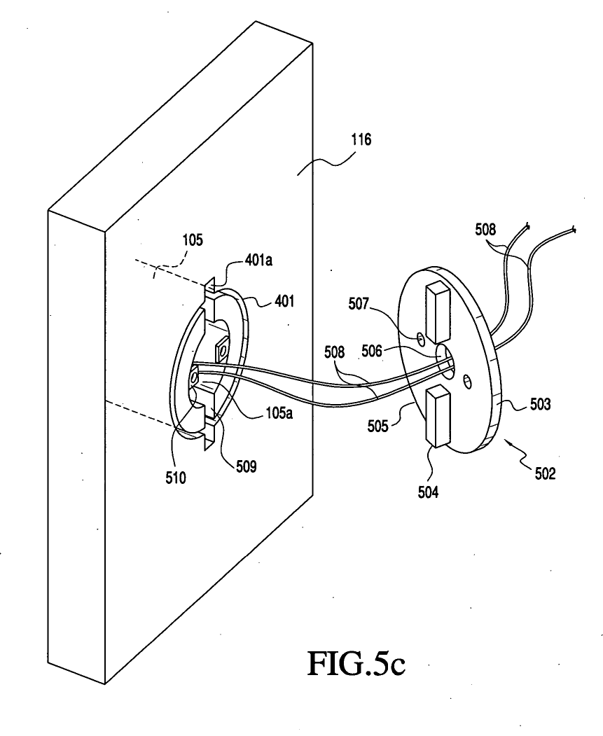

It is important that the tanning bed not rotate independently relative to the axle. The easiest way to do this is to bolt a locking plate to both the axle 105 and support 116 (See FIG. 5a; please note that the size of the axle and all associated parts is greatly exaggerated relative to support 116, for the sake of clarity.). Also, axle 105 may be provided with a longitudinal slot, a longitudinal tab, or a flattened surface 105b that interacts with a corresponding tab, slot, or flat surface 401b in hole 401 of support 116. A locking plate 502 may then be bolted to the axle using one or more threaded bolt-receiving elements 510 mounted inside the bore of axle 105, and one or more threaded bolt-receiving holes 511 through support 116. The locking plate may be unbolted from the axle and the support 116, allowing the tanning bed assembly to be removed from the framework 100 by sliding support 116 off of axle 105. This greatly eases transportation of the tanning bed assembly by allowing the tanning bed to be moved as two smaller, lighter units, rather than as a single bulky unit. Also, after a tanning bed assembly is removed from axle 105, it may be replaced with a new tanning bed assembly without requiring the replacement of framework 100.

FIG. 5b shows another method of securing tanning bed support 116 to axle 105 The hole 401 through support 116 is positioned over axle 105. Cables 508 run through the longitudinal bore 105a of axle 105, and then through a hole 506 through the center of a locking plate 502. Plate 502 has an outer plate 503 having two bolt-receiving holes 507 therethrough. On the inner surface of plate 503, which is placed against support 116, two tabs 504 are positioned. These tabs fit into notches 401a in support 116, rotationally locking the support 116 relative to plate 502. Bolts may then be passed through bolt-receiving holes 507 in plate 502 and screwed into threaded holes 501 in axle 105, rotationally locking the axle 105 relative to plate 502. Thus, the axle and the axle support are forced to rotate as a unit. The rotational stability of the unit may be further secured if axle 105 has at least one flattened surface 105b. In this case, an arc-shaped inner plate 505 may be mounted on the inner surface of plate 503, where inner plate 503 has the same radius of curvature as axle 105. When plate 502 is positioned against support 116 and bolted into position, a flat surface of arc-shaped inner plate 505 is positioned against flattened surface 105b of axle 105, making it impossible for the axis and the plate 502 to rotate independently. Wires 508 are then connected to the lighting system in the canopy unit 402 and base unit 405, and used to power the UV lights. Additional wires may be used to power cooling fans (not shown in the figures) to control heat buildup in units 402 and 405

An alternate mechanism for securing tanning bed support 116 to axle 105 is shown in FIG. 5c. In this system, plate 502 has an outer plate 503 having two bolt-receiving holes 507 therethrough. On the inner surface of plate 503, two tabs 504 are positioned. Axle 105 is a simple tube having two longitudinal slots 509 in its wall. Two members 510 having threaded bolt-receiving holes may be welded to the inner surface of the bore 105a through axle 105; also, two threaded bolt-receiving holes may be formed in support 116, adjacent to axle-receiving hole 401a. Plate 502 is then used to cover hole 401 in support 116, with tabs 504 interlocking with slots 509 in the axle and with notches 401a in support 116. This rotationally locks the axle relative to the support 116. Plate 502 may then be bolted into position.

Removal of the bolts from plate 502 allows plate 502 to be removed from the axle 105. Cables 508 may then be detached from the UV light fixtures and other electrical components in the tanning bed, and the tanning salon proprietor may then remove the tanning bed from the support frame 100 by sliding the tanning bed longitudinally off of axle 105. These two units may be more easily transported from one location to another, because of their reduced bulk as compared to prior art tanning beds, which cannot be disassembled. Also, the support frame 100 and the tanning bed, once separated, are more easily able to fit through a standard door. If the tanning bed puts out a new model of tanning bed with significant upgrades, the old tanning bed may be readily removed from axle 105 and replaced with a new tanning bed. There is no need to replace the support frame 100. Replacement of the tanning bed is thus made easier since only a portion of the apparatus needs to be moved. Also, since the support frame 100 may be used with the new tanning bed, the tanning salon proprietor does not have to buy a complete tanning bed apparatus, greatly reducing the cost of replacements and/or upgrades of tanning beds.

The motor may be placed under computer control, if desired. Prior to a tanning session, the tanning salon proprietor or the customer would input a desired tanning position to computer, where the tanning position is horizontal, vertical, or somewhere in between. The computer would then activate the motor and cause the motor to rotate the tanning bed until the desired tanning position is achieved. The computer may also be used to control the power supply to the UV lamps in the tanning bed. The computer would be pre-programmed with the maximum safe exposure time to the light from the UV lamps. At the start of the tanning session, power to the UV lamps would be activated under computer control. The computer then monitors the exposure time to the UV lamps, and automatically shuts off power to the UV lamps when the maximum safe exposure time is reached.

Additional features that may be added to the tanning bed system include a manual crank 119 which would allow a tanning salon proprietor, or a tanning salon customer, to manually rotate axle 105. This would primarily be needed in the event of a loss of power or of damage to the motor; however, the crank may be used at any time if the user so chooses. This crank would, for example be connected to shaft 115 in the embodiment of FIG. 1b. Shaft 115 may then be divided into two parts, a first shaft directly engaged by motor 108 and a second shaft carrying gear 112. A series of projections, or splines, on the shaft engaged by the motor fit into slots on the corresponding gear-bearing shaft, enabling both to rotate together upon rotation of the motor 108. When a user wishes to rotate the tanning bed manually, the splines disengage the slots on the gear-bearing shaft, allowing the gear-bearing shaft to rotate independently of the motor. The tanning bed may then be manually rotated by rotating crank 119. Disengagement of the slots on the gear-bearing shaft by the splines may be controlled in a number of ways. The splines may be disengaged in response to a manually operated switch. The splines may also engage the slots on the gear-bearing shaft under computer control. The splines may be engaged automatically during computer-controlled rotation of the tanning bed and during a computer-timed tanning session, when the UV lights are on. Also, the tanning bed may be counterbalanced about its rotational axis to increase the ease of manual rotation.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

1. A rotatably mounted tanning bed system, said system comprising:
a) a support frame;
b) an axle rotatably mounted to said support frame;
c) a tanning bed mounted to said axle; and
d) a motor; and
e) a means for transferring rotational motion from said motor to said axle;
said motion-transferring means being adapted to reversibly rotate the axle between a first position and a second position, wherein said tanning bed is in a horizontal position when the axle is in said first position, and said tanning bed is in a vertical position when the axle is in said second position;
wherein said axle, said motor, and said motion-transferring means are the only moving parts present in said system.

2. The tanning bed system of claim 1, wherein:
the motion-transferring means is adapted to reversibly rotate the axle from said first position through at least one intermediate position to said second position, said at least one intermediate position being between said first position and said second position;
wherein rotation of said motor may be stopped at said first position, said at least one intermediate position, or said second position.

3. The tanning bed system of claim 2, wherein said at least one intermediate position may be selected by the user.

4. The tanning bed system of claim 2, wherein said at least one intermediate position may be selected by the user from a plurality of positions preprogrammed into a computer controlling operation of the motor.

5. The tanning bed system of claim 1, wherein the tanning bed comprises a bed adapted to support the weight of an occupant, and a canopy, said canopy facing said bed so that said occupant may position himself between said canopy and said bed.

6. The tanning bed system of claim 5, wherein UV lamps are mounted in the canopy, said UV lamps being adapted to irradiate said occupant.

7. The tanning bed of claim 5, wherein a first set of UV lamps is mounted in the canopy, and a second set of UV lamps is mounted beneath the weight-supporting bed, said second set of UV lamps being adapted to shine on an occupant's body through the weight-supporting bed.

8. The tanning bed system of claim 6, wherein the axle has a bore, said bore allowing electrical wires to pass therethrough, wherein said electrical wires provide power to said UV lamps.

9. The tanning bed of claim 1, wherein said motion-transferring means comprises a set of gears, said set of gears allowing the motor to rotate the axle indirectly.

10. The tanning bed system of claim 9, wherein the motor rotates a first gear, and rotation of said first gear rotates a main gear mounted on said axle.

11. The tanning bed of claim 10, wherein said first gear is a first spur gear and said main gear is a second spur gear, said second spur gear being mounted on said axle so that said second spur gear and said axle are coaxial.

12. The tanning bed system of claim 10, wherein said first gear is a worm gear having a spiral thread, and said main gear is a spur gear having teeth that mesh with said spiral thread, said spur gear being mounted on said axle so that said spur gear and said axle are coaxial.

13. The tanning bed system of claim 1, wherein said motion-transferring means allows the motor to rotate the axle directly.

14. The tanning bed system of claim 1, wherein the tanning bed is removably mounted to said axle.

15. The tanning bed system of claim 14, further comprising a means for preventing the tanning bed from rotating independently of said axle.

16. The tanning bed system of claim 14, further comprising a means for preventing the tanning bed from rotating longitudinally on said axle.

17. The tanning bed system of claim 1, further comprising a means for minimizing friction between said support frame and said axle.

18. The tanning bed system of claim 17, wherein said means for minimizing friction is a bearing.

19. The tanning bed system of claim 17, wherein said means for minimizing friction is a roller bearing.

20. The tanning bed system of claim 17, wherein said means for minimizing friction is a radial ball bearing having an outer ring mounted to said support frame, an inner ring mounted to said axle, and a plurality of balls therebetween.

21. The tanning bed system of claim 17, wherein:
said support frame further comprises a tubular axle support having a bore, where said axle passes through the bore of said axle support; and
said means for minimizing friction comprises a low-friction fluoropolymer material positioned between the axle and the axle support.

22. The tanning bed system of claim 1, wherein the support frame comprises a vertical support and a horizontal base, said horizontal base comprising a trench adapted to receive a first end of said tanning bed when said tanning bed is in a vertical position.

23. The tanning bed system of claim 22, wherein said horizontal base additionally comprises a vertical surface adapted to prevent rotation of the axle from moving the tanning bed past said vertical position.

24. The tanning bed system of claim 23, wherein said horizontal base additionally comprises a shock absorbing means to reduce the force of an impact between said tanning bed and said vertical surface.

25. The tanning bed system of claim 24, wherein said shock absorbing means comprises a pneumatic damper.

26. A rotatably mounted tanning bed system, said system comprising:
a) a support frame;
b) an axle rotatably mounted to said support frame;
c) a tanning bed removably mounted to said axle; and
d) a motor; and
e) a means for transferring rotational motion from said motor to said axle;
said motion-transferring means being adapted to reversibly rotate the axle from a first position to a second position, wherein said tanning bed is in a horizontal position when the axle is in said first position, and said tanning bed is in a vertical position when the axle is in said second position.

27. The tanning bed system of claim 26, wherein the axle has a bore, said bore allowing electrical wires to pass therethrough, wherein said electrical wires provide power to said UV lamps.

28. The tanning bed of claim 26, wherein the motor rotates the axle indirectly.

29. The tanning bed system of claim 28, wherein the motor rotates a first gear, and rotation of said first gear rotates a main gear mounted on said axle.

30. The tanning bed of claim 29, wherein said first gear is a first spur gear and said main gear is a second spur gear, said second spur gear being mounted on said axle so that said second spur gear and said axle are coaxial.

31. The tanning bed system of claim 29, wherein said first gear is a worm gear having a spiral thread, and said main gear is a spur gear having teeth that mesh with said spiral thread, said spur gear being mounted on said axle so that said spur gear and said axle are coaxial.

32. A framework for supporting a rotatably mounted tanning bed, comprising:
a) a support frame;
b) an axle rotatably mounted to said support frame, said axle being adapted to non-rotatably support a tanning bed; and
c) a motor;
d) a means for transferring rotational motion from said motor to said axle;
said motion-transferring means adapted to reversibly rotate the axle from a first position to a second position, wherein said tanning bed is in a horizontal position when the axle is in said first position, and said tanning bed is in a vertical position when the axle is in said second position;
wherein said axle, said motor, and said motion-transferring means are the only moving parts present in said framework.

33. A rotatably mounted tanning bed system, said system comprising:
a) a support frame;
b) an axle rotatably mounted to said support frame;
c) a tanning bed mounted to said axle;
d) a system of gears for rotating said axle; and
e) a means for driving said system of gears;
said driving means being adapted to reversibly rotate the axle between a first position and a second position, wherein said tanning bed is in a horizontal position when the axle is in said first position, and said tanning bed is in a vertical position when the axle is in said second position;
wherein said axle, said motor, and said motion-transferring means are the only moving parts present in said system.

34. The tanning bed system of claim 33, wherein:
the driving means is adapted to reversibly rotate the axle from said first position through at least one intermediate position to said second position, said intermediate position being between said first position and said second position;
wherein rotation of said motor may be stopped at said first position, said at least one intermediate position, or said second position.

35. The tanning bed system of claim 33, wherein:
the driving means is a computer-controlled motor.

36. The tanning bed system of claim 33, wherein:
the driving means is manually driven.
